(19) United States
(12) Patent Application Publication
(10) Pub. No.: US 2026/0016409 A1
(43) Pub. Date: Jan. 15, 2026

(54) LASER-BASED WIRELESS METHANE MONITORING SYSTEM BASED ON PULSE-DRIVEN VERTICAL-CAVITY SURFACE-EMITTING LASER AND MONITORING METHOD THEREOF

(71) Applicant: SHANDONG MICRO-SENSOR PHOTONICS CO. LTD., Jinan (CN)

(72) Inventors: Tongyu LIU, Jinan (CN); Yanong NING, Jinan (CN); Liang YAN, Jinan (CN); Chengwu GU, Jinan (CN); Dehu LI, Jinan (CN)

(21) Appl. No.: 19/337,044

(22) Filed: Sep. 23, 2025

Related U.S. Application Data
(63) Continuation of application No. PCT/CN2024/109230, filed on Aug. 1, 2024.

(30) Foreign Application Priority Data
Jul. 8, 2024 (CN) 2024109087273

(51) Int. Cl. G01N 21/39; G01N 33/00

(52) U.S. Cl. CPC G01N 21/39; G01N 33/0036; G01N 2021/399

(57) ABSTRACT

An ultra-low power consumption laser-based wireless methane monitoring system based on a pulse-driven vertical-cavity surface-emitting laser (VCSEL), wherein when the system is in a normal operation, a microcontroller unit (MCU): automatically wakes up a corresponding laser-based methane node detection device to measure ambient temperature; according to measured different ambient temperature values, performs detection of methane concentration by controlling wavelength of laser beam emitted by VCSEL light source chip to be scanned at different absorption peak wavelengths; and judges methane concentration value is in a preset safety range or not; wherein, if yes, switches the corresponding device to sleep and wakes it up when a preset sleep period ends, to detect methane concentration again; and, if the methane concentration value is always within the preset safety range, the device continuously operates alternately and repeatedly between “wake-up” and “sleep” modes; if not, makes an alarm prompt and uploads methane concentration data to a monitoring terminal.

### CROSS-REFERENCE TO RELATED APPLICATIONS

The present invention claims priority benefits to China patent application number 202410908727.3, entitled “An Ultra-low Power Consumption Wireless Methane Monitoring System Based on Pulse-driven Vertical-cavity Surface-emitting Laser and Monitoring Method Thereof”, filed on Jul. 8, 2024 with China National Intellectual Property Administration, the entire contents of which are incorporated herein by reference and form a part of the present invention for all purposes.

### TECHNICAL FIELD

The present invention belongs to the field of safety monitoring leakage of methane or natural gas, and particularly relates to an ultra-low power consumption laser-based wireless methane monitoring system based on a pulse-driven vertical-cavity surface-emitting laser (VCSEL) and a monitoring method thereof.

### BACKGROUND

The statements in this section merely provide background information related to the present invention and do not necessarily constitute prior art.

Due to the existing cross-interference problem of methane alarm devices based on electrochemical or catalytic combustion, the technology of tunable diode laser absorption spectroscopy (TDLAS) has more prominent advantages.

However, in many practical application scenarios, the methane detection and alarm devices are required to be powered by batteries, and the measurement frequency of methane concentration is required to be once in a few seconds or more than ten seconds, and the work life time of each battery in the methane sensor needs to be several years or even nearly ten years without replacing the battery module. These application requirements are difficult to be met with the use of a conventional TDLAS methane sensor using a DFB (Distributed Feedback) laser as the light source, due to its large power consumption, and therefore, the above-mentioned application requirements pose a technical challenge to the existing related art.

### SUMMARY

In order to solve the mentioned technical problems existing in the background art, the present invention provides an ultra-low power consumption wireless methane monitoring system based on a pulse-driven VCSEL and a monitoring method thereof, which may not only measure methane concentration once in several seconds or more than ten seconds, but also ensure that the battery used to power the node detection device of methane does not need to replace over several years or even ten years.

In order to achieve the above object, the present invention adopts the following technical solutions.

A first aspect of the present invention provides an ultra-low power consumption laser-based wireless methane monitoring system based on a pulse-driven VCSEL, comprising a group of laser-based methane node detection devices powered by batteries, wherein each laser-based methane node detection device comprises a pulse-driven VCSEL-based methane detection module, and the pulse-driven VCSEL-based methane detection module comprises a microcontroller unit (MCU) control module; wherein,
the MCU control module is configure to:
(1) automatically wake up a corresponding pulse-driven VCSEL-based methane detection module when the ultra-low power consumption laser-based wireless methane monitoring system is in a normal operation; (2) receive an ambient temperature at a current moment from the corresponding pulse-driven VCSEL-based methane detection module; (3) according to the received different ambient temperature, control a drive current of a VCSEL light source chip, so that a wavelength of a laser beam emitted by the VCSEL light source chip is scanned at different absorption peak wavelengths, to carry out a measurement to methane concentration; (4) perform an inversion calculation based on a calibration value to obtain a value of the methane concentration; and (5) judge whether the obtained value of the methane concentration is in a preset safety range, which comprises:
if yes, to switch a state of the corresponding pulse-driven VCSEL-based methane detection module to a “sleep” mode until the end of a preset sleep period, then wake up the corresponding pulse-driven VCSEL-based methane detection module once again to perform the detection of the methane concentration; and, if the obtained value of the methane concentration through the detection is always within the preset safety range, to control the pulse-driven VCSEL-based methane detection module to continuously operate alternately and repeatedly in a “wake-up” mode and the “sleep” mode;
if not, which indicates that the obtained value of the methane concentration through the detection exceeds the preset safety range, to send out an alarm prompt information.

As an implementation mode, the pulse-driven VCSEL-based methane detection module further comprises a gas absorption cell optical path, wherein the gas absorption cell optical path comprises a chip-on-board (COB) circuit board, a collimating concave mirror, a focusing concave mirror and an elliptic concave mirror; wherein,
the VCSEL light source chip and a photodetector chip are respectively fixed at preset positions on the COB circuit board, a light outlet of the VCSEL light source chip is positioned at a focus of the collimating concave mirror, a photosensitive surface of the photodetector chip is positioned at a focus of the focusing concave mirror, and the VCSEL light source chip and the photodetector chip are further respectively connected to the MCU control module; wherein,
the light beam emitted from the light outlet of the VCSEL light source chip is reflected by the collimating concave mirror to form a collimated light beam parallel to a plane the COB circuit board, the collimated light beam is reflected by the elliptic concave mirror and then is incident on the focusing concave mirror, and the incident light beam is reflected by the focusing concave mirror to focus on the photosensitive surface of the photodetector chip, and the photodetector chip converts an optical signal into an electrical signal and then transmits the electrical signal to the MCU control module.

As an implementation mode, the VCSEL light source chip and the photodetector chip may be replaced by a transistor outline-can (TO-can) device.

As an implementation mode, the collimating concave mirror and the focusing concave mirror are provided with a rotating parabolic concave surface shape.

As an implementation mode, the ultra-low power consumption laser-based wireless methane monitoring system further comprises a monitoring terminal, wherein the monitoring terminal is connected with the laser-based methane node detection devices through a wireless gateway; and
the monitoring terminal is configured to: receive a current ambient temperature value, the value of the methane concentration, an alarm status and an alarm node position information sent from each of the laser-based methane node detection devices at each preset time interval, and summarize, store and display the information.

As an implementation mode, the each of the laser-based methane node detection devices further comprises a wireless data communication module and a battery driving module, wherein the wireless data communication module and the battery driving module are electrically connected with the MCU control module, the wireless data communication module uploads data of the methane concentration to the monitoring terminal, and the battery driving module supplies power to the laser-based methane node detection device.

As an implementation mode, the each of the laser-based methane node detection devices further comprises an acousto-optic alarm module, wherein the acousto-optic alarm module is connected to the pulse-driven VCSEL-based methane detection module, and if the measured value of the methane concentration through the detection exceeds the preset safety range, the acousto-optic alarm module makes an alarm prompt.

As an implementation mode, the battery driving module may be a D-type battery with a model number of CR34615, and the capacity thereof is 19 Ah.

As an implementation mode, the pulse-driven VCSEL-based methane detection module further comprises an embedded firmware, wherein the embedded firmware is written into a nonvolatile memory of the MCU control module, and after the methane concentration detection is performed by using the different absorption peak spectra, the embedded firmware may automatically search absorption peaks and perform the inversion calculation to output the value of the methane concentration.

In order to solve the above mentioned technical problems, a second aspect of the present invention provides a methane monitoring method by using the ultra-low power consumption laser-based wireless methane monitoring system based on a pulse-driven VCSEL according to the first aspect, comprising the following steps:
the MCU control module wakes up the pulse-driven VCSEL-based methane detection module corresponding thereto, to measure the ambient temperature at the current moment;
the MCU control module receives the measured ambient temperature at the current moment, then according to the measured ambient temperature value, controls the drive current of the VCSEL light source chip, so that the wavelength of the laser beam emitted by the VCSEL light source chip is scanned at different absorption peak wavelengths, to carry out the measurement of the methane concentration, then performs the inversion calculation based on the calibration value to obtain the value of the methane concentration; and
the MCU control module executes a judgment that whether the obtained value of the methane concentration is within the preset safety range or not, wherein:
if yes, switches the state of the corresponding pulse-driven VCSEL-based methane detection module to the “sleep” mode until the end of the preset sleep period, wakes up the corresponding pulse-driven VCSEL-based methane detection module once again to perform the detection of the methane concentration; and, if the obtained value of the methane concentration through the detection is always within the preset safety range, controls the pulse-driven VCSEL-based methane detection module to continuously operate alternately and repeatedly in a “wake-up” mode and the “sleep” mode;
if not, which indicates that the obtained value of the methane concentration through the detection exceeds the preset safety range, controls a corresponding laser-based methane node detection device to make an alarm prompt.

Compared with the prior art, the invention has the advantages that:
1. According to the present invention, the laser-based methane node detection device uses the VCSEL as the light source, and may adjust a scanning current of the laser according to different ambient temperatures and make a measurement with the use of different methane absorption peak spectra, so that the laser may work in different temperature ranges, which not only expands the working temperature range of the sensor, but also reduces a power consumption by removing the need of a thermoelectric cooler (TEC).
2. In order to protect the VCSEL from the impact of pulse current, the MCU control module adopts a current driving scheme of the VCSEL light source chip comprising a slow-start and a slow-turn off modes when the MCU automatically switches the laser-based methane node detection device between “wake-up”/“sleep” modes. Through the pulse driving circuit which may automatically be asleep and be woke up by the MCU control module, so as the laser-based methane node detection device may detect the methane concentration at regular intervals, and because the laser-based methane node detection device is in a sleep mode state during the time interval of every two detection, the operation power dissipation of the laser-based methane node detection device is further reduced.
3. Since the MCU control module in each the laser-based methane node detection device can independently detect and judge the value of the methane concentration at the node, it can ensure that the value of the methane concentration at the node location is always kept within a safe range. If not, an alarm module in the laser-based methane node detection device will alarm at the node location to ensure that the methane concentration at the node location is detected and alarmed in time. The MCU control module also sends the collected data and judgment results to the background monitoring terminal at the same time, and the background monitoring terminal displays the collected data, alarm status and alarm position points, so that the monitoring terminal can monitor and manage these application scenarios in an all-day, all-weather and all-process manner to prevent gas leakage and ensure the safety of life and property.
4. According to the present invention, the laser-based methane node detection devices are driven by the battery, and the detected concentration values can be transmitted in the wireless communication method, thus the laser-based methane node detection devices can be installed in various application scenarios where there is no power supply or is difficult to connect to power lines, and then a group of the laser-based methane node detection devices are connected into a larger methane wireless detection network by using a wireless node aggregation gateway and connected with a background monitoring terminal; in this way, the background monitoring terminal can monitor and manage different application scenarios such as natural gas pipelines, buildings with gas, residential buildings and other places for a long time, and timely monitor and alarm natural gas leakage accidents to ensure the safety of life and property.

Additional aspects of the present invention will be set forth in part in the description which follows, and in part will be obvious from the description, or may be learned by practice of the present invention.

### BRIEF DESCRIPTION OF THE DRAWINGS

The accompanying drawings constituting a part of the present invention are used to provide a further understanding of the present invention. The exemplary examples of the present invention and descriptions thereof are used to explain the present invention, and do not constitute an improper limitation of the present invention.

FIG. 1 is a schematic diagram of an ultra-low power consumption laser-based wireless methane monitoring system based on pulse-driven VCSEL methane node detection devices according to examples of the present invention;

FIG. 2 is a functional structure diagram of a laser-based methane node detection device according to examples of the present invention;

FIG. 3 is a functional structure diagram of a pulse-driven VCSEL-based methane detection module according to examples of the present invention;

FIG. 4 is a schematic diagram of a gas absorption cell optical path using concave mirrors with a curved reflecting surface according to examples of the present invention;

FIG. 5 is a process diagram of a wireless monitoring method for a laser-based methane node detection device according to examples of the present invention;

FIG. 6 is a schematic diagram of the gas absorption cell optical path using a TO-can laser and a detector according to examples of the present invention.

### DETAILED DESCRIPTION

The present invention will now be further described with reference to the accompanying drawings and examples.

It should be pointed out that the following detailed descriptions are all illustrative and are intended to provide further descriptions of the present invention. Unless otherwise specified, all technical and scientific terms used in the present invention have the same meanings as those usually understood by a person of ordinary skill in the art to which the present invention belongs.

It should be noted that the terms used herein are merely used for describing specific implementations, and are not intended to limit exemplary implementations of the present invention. As used herein, the singular form is also intended to include the plural form unless the context clearly dictates otherwise. In addition, it should further be understood that, terms “comprise/comprising” and/or “include/including” used in this specification indicate that there are features, steps, operations, devices, components, and/or combinations thereof.

In the present invention, terms such as “connected with” and “connected to” should be broadly understood, indicating that they can be fixed connections, integrated connections, or detachable connections; It can be directly connected or indirectly connected through an intermediate medium. For relevant scientific research or technical personnel in this field, the specific meanings of the above terms in the present invention can be determined according to specific circumstances, and cannot be understood as limitations on the present invention.

### Example 1

The present example provides an ultra-low power consumption laser-based wireless methane monitoring system based on a pulse-driven VCSEL, comprising:

As shown in FIG. 1, the monitoring system comprises a group of laser-based methane node detection devices powered by batteries and a background monitoring terminal;
the plurality of the laser-based methane node detection devices can be installed in various application scenarios where on power supply available or difficult to connect with power supply lines, and the group of the laser-based methane node detection devices can be connected into a larger methane detection network by using a wireless node aggregation gateway; the gateway is connected with the background monitoring terminal, and the monitoring terminal collects, stores and displays current environmental temperature values, concentration values of methane, alarm states and alarm position points of the group of the laser-based methane node detection devices, so as to realize long-term monitoring and management of the group of the laser-based methane node detection devices installed in different application scenarios, such as long-term monitoring and management of natural gas pipelines, buildings and halls with gas, residential houses and other places, timely monitor and alarm natural gas leakage accidents, and ensure the safety of life and property.

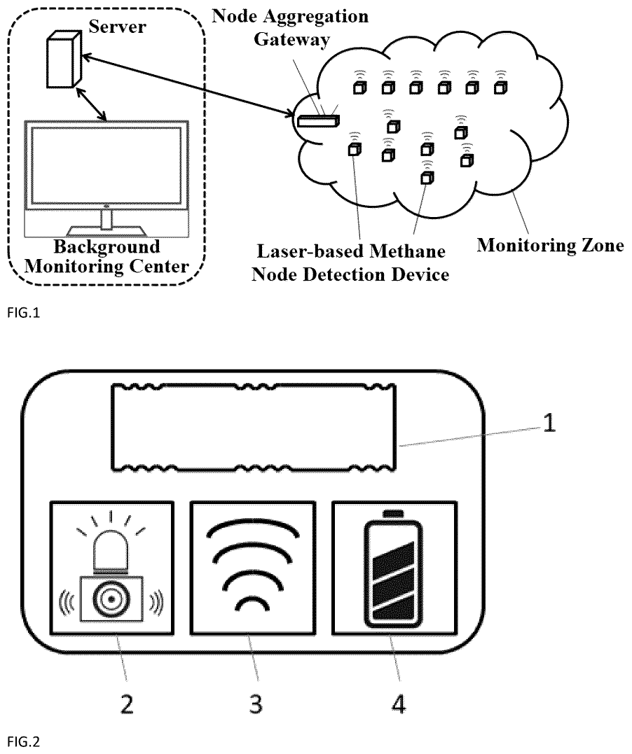

As shown in FIG. 2, each of the group of the laser-based methane node detection devices comprises a pulse-driven VCSEL-based methane detection module 1, an acousto-optic alarm module 2, a wireless data communication module 3 and a battery driving module 4.

The wireless data communication module 3 and the battery driving module 4 are electrically connected with the pulse-driven VCSEL-based methane detection module 1, methane concentration data is uploaded to the monitoring terminal through the wireless data communication module 3, and power is supplied to the laser-based methane node detection device through the battery driving module 4.

In the present example, the wireless data communication module may be a 3G, 4G, GPRS or WIFI wireless data communication module. The driving battery is a D-type battery with a model number of CR34615 and a capacity of 19 Ah.

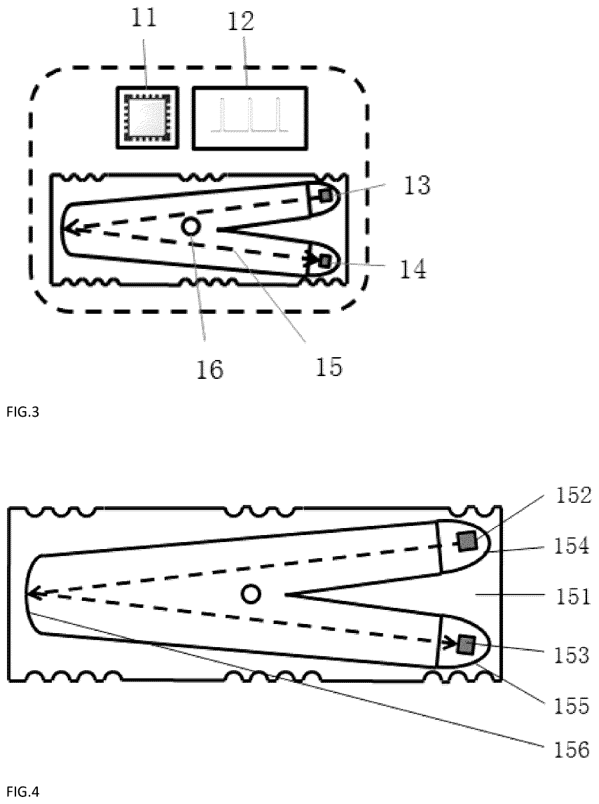

As shown in FIG. 3, the pulse-driven VCSEL-based methane detection module 1 comprises a MCU control module 11, a pulse driving circuit 12 which is capable of automatically switching between an asleep and waking up mode, a VCSEL light source zone 13, a photodetector zone 14, a lens-free gas absorption cell optical path 15 composed of curved reflective surfaces, a temperature and pressure sensor 16, and an embedded firmware 17 which is capable of automatically searching absorption peaks and calculating concentration values of methane.

It should be noted that in the present example, the pulse-driven VCSEL-based methane detection module utilizes the characteristics of low power dissipation, tunability and narrow line-width of the VCSEL to select specific absorption spectra of different kinds of gases to be detected in different temperature ranges for detection, which not only eliminates interference from spectra of other kinds of gases, but also broadens the working temperature range of the pulse-driven VCSEL-based methane detection module. In addition, the pulse-driven VCSEL-based methane detection module of the present example has high accuracy in a certain temperature range (usually −30° C. to 70° C.).

As shown in FIG. 4, the lens-free gas absorption cell optical path 15 composed of curved reflecting surfaces comprises a COB circuit board 151, a VCSEL light source chip 152 disposed in the VCSEL light source zone 13, a photodetector chip 153 disposed in the photodetector zone 14, a collimating concave mirror 154, a focusing concave mirror 155 and an elliptic concave mirror 156, wherein the two concave mirrors are made of rotating parabolic reflecting mirrors.

The VCSEL light source chip 152 and the photodetector chip 153 are respectively fixed at preset positions of the COB circuit board 151, a light outlet of the VCSEL light source chip 152 is located at the focal point of the collimating concave mirror 154, and the photosensitive surface of the photodetector chip 153 is located at the focal point of the focusing concave mirror 155. Then, bonding pads of the VCSEL light source chip 152 and the photodetector chip 153 are respectively connected to the circuits on the COB circuit board through golden wires, so that the VCSEL light source chip 152 is connected to the pulse driving circuit 12 controlled by the MCU control module, and the photodetector chip 153 is connected to the MCU control module through an automatic gain circuit and an analog-to-digital converter (ADC) sampling circuit.

A laser light beam emitted from the light outlet of the VCSEL light source chip 152 is reflected by the collimating concave mirror 154 to form a collimated light beam parallel to a plane the COB circuit board 151, the collimated light beam is reflected by the elliptic concave mirror 156 and then is incident on the focusing concave mirror 155, and the incident light beam is reflected by the focusing concave mirror 155 to focus on the photosensitive surface of the photodetector chip 153, and the photodetector chip 153 converts the optical signal into an electrical signal, and the electrical signal is amplified and converted through the ADC, and then is input to the MCU control module.

Because no lens is adopted in the optical path design of the gas absorption cell optical path, i.e. no collimating lens is adopted in front of the light outlet of the VCSEL light source chip and no focusing lens is adopted in front of the photodetector chip, interference background noise introduced by specular reflection light of the lenses can be effectively avoided, and the effect of increasing detection sensitivity is achieved.

As shown in FIG. 5, when the ultra-low power consumption laser-based wireless methane monitoring system is in normal operation, the MCU control module in each of the laser-based methane node detection devices automatically wakes up the laser-based methane node detection device corresponding thereto; at this time, the MCU control module controls the temperature sensor to measure the ambient temperature at the current moment, and for different measured ambient temperature values, controls (adjusts) the drive current of the VCSEL light source chip, so that the wavelength of the laser beam emitted by the VCSEL light source chip is scanned at different absorption peak wavelengths, to detect the methane concentration, and the embedded firmware automatically searches absorption peaks and calculates the measured value of the methane concentration by inversion; after the VCSEL light source chip enters a stable working state, the methane concentration in the environment, where a present laser-based methane node detection device is located, is sampled and measured, and the MCU control module judges the value of the methane concentration collected at the place (node) where the present laser-based methane node detection device is located.

If the value of the methane concentration detected by one laser-based methane node detection device is within a preset safety range, the MCU control module therein switches the corresponding present laser-based methane node detection device into a “sleep” mode; when the preset sleep period ends, the MCU control module automatically wakes up the corresponding laser-based methane node detection device to repeatedly detect methane concentration; if the obtained value of the methane concentration through the detection is always within the preset safety range, the corresponding laser-based methane node detection device alternately and repeatedly operates between the “waking-up” mode and “sleep” mode; simultaneously, the MCU control module starts the wireless data communication module to upload the collected data to the background monitoring terminal once after a preset time interval, such as several hours or 24 hours, so that the monitoring terminal can monitor and record the operation state of the laser-based methane node detection device.

If the value of the methane concentration detected by one laser-based methane node detection device is greater than a preset alarm value, the MCU control module of the corresponding laser-based methane node detection device starts the acousto-optic alarm module to perform field alarm prompt at the node, and uploads the collected methane concentration data to a background monitoring terminal through the wireless data communication module, or starts a linkage device such as an electric ball valve to perform corresponding processing of closing the gas pipeline.

Because the MCU control module of each of the laser-based methane node detection devices can independently detect and judge the value of the methane concentration at the node, the value of the methane concentration at the node can be guaranteed to be kept within a safe range all the time; if not, the alarm module of the laser-based methane node detection device is controlled to alarm at the node, so that the methane concentration at the node can be timely detected and alarmed; the MCU control module also uploads the collected data and judgment results to the background monitoring terminal at the same time, and displays the collected data, alarm status and alarm position points in the background monitoring terminal, so that the monitoring terminal can monitor and manage these application scenarios in an all-day, all-weather and all-process manner to prevent gas leakage and ensure the safety of life and property.

Since that laser-based methane node detection device adopt the VCSEL as the light source, the laser-based methane node detection device can adjust scanning current of the laser according to different environmental temperatures and adopt different methane absorption peak spectra for detection, so that the laser can work in different temperature interval, thus not only expanding the working temperature range of the sensor, but also reducing power dissipation of temperature control of TEC because the VCSEL laser does not need TEC.

In order to protect the VCSEL light source chip from the impact of pulse current, the MCU control module adopts a current driving scheme of the VCSEL light source chip comprising a slow-start and a slow-turn off when automatically wakes up the laser-based methane node detection device. Through the pulse driving circuit which can automatically be asleep and be woke up by the MCU control module, so as the laser-based methane node detection device can detect the methane concentration at regular intervals, and because the laser-based methane node detection device is in a closed state in the time interval of every two detection, the operation power dissipation of the laser-based methane node detection device is further reduced.

### Example 2

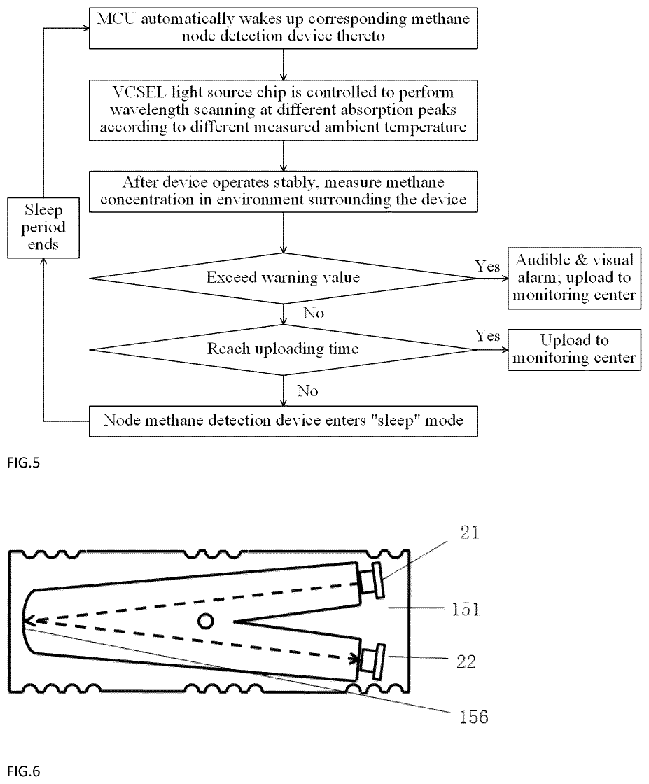

The present example provides an ultra-low power consumption laser-based wireless methane monitoring system based on pulse-driven VCSEL and driven by batteries, which is described in detail with reference to FIG. 6 in addition to the schemes shown in FIGS. 1 to 5 above. As shown in FIG. 6, a TO-can device for detection is installed in the VCSEL light source zone 13 and the photodetector zone 14 for replacing. The TO-can device comprises a VCSEL laser 21 with a collimating lens, a COB circuit board 151, a detector 22 with a focusing lens, and an elliptical mirror 156. The specific working principle is the same as that of the gas absorption cell optical path 15.

The advantage of the replacement solution of the present example is: the VCSEL lasers or detectors of different types or manufacturers can be selected to realize the function of the methane wireless monitoring system; and the disadvantage thereof is: the measurement accuracy is reduced due to interference noise introduced by lens surface reflection due to lens adoption in the optical path.

### Example 3

The present example provides a methane monitoring method by using the ultra-low power consumption laser-based wireless methane monitoring system based on a pulse-driven VCSEL according to the Example 1 or Example 2, comprising the following steps:
the MCU control module measures the ambient temperature at the current moment, for different ambient temperature, controls the drive current of the VCSEL light source chip, so that the wavelength of the laser beam emitted by the VCSEL light source chip is scanned at different absorption peak wavelengths, to carry out the measurement to the methane concentration, then performs the inversion calculation based on the calibration value to obtain the value of the methane concentration; and
the MCU control module judges that whether the obtained value of the methane concentration is in the preset safety range or not, wherein:
if yes, switches the state of the corresponding pulse-driven VCSEL-based methane detection module to the “sleep” mode until the end of the preset sleep period, wakes up the corresponding pulse-driven VCSEL-based methane detection module once again to perform the detection of the methane concentration; and, if the obtained value of the methane concentration through the detection is always within the preset safety range, controls the pulse-driven VCSEL-based methane detection module to continuously operate alternately and repeatedly in a “wake-up” mode and the “sleep” mode;
if not, which indicates that the obtained value of the methane concentration through the detection exceeds the preset safety range, makes an alarm prompt.

The foregoing descriptions are merely preferred embodiments of the present invention but are not intended to limit the present invention. A person skilled in art may make various alterations and variations to the present invention. Any modification, equivalent replacement, or improvement made within the spirit and principle of the present invention shall fall within the protection scope of the present invention.

1. A laser-based wireless methane monitoring system based on a pulse-driven vertical-cavity surface-emitting laser (VCSEL), comprising:
a group of laser-based methane node detection devices powered by batteries, wherein each of the laser-based methane node detection devices comprises a pulse-driven VCSEL-based methane detection module, and the pulse-driven VCSEL-based methane detection module comprises a microcontroller unit (MCU) control module; wherein,
the MCU control module is configure to:
automatically wake up a corresponding pulse-driven VCSEL-based methane detection module when the laser-based wireless methane monitoring system is in a normal operation, receive a measured ambient temperature at a current moment,
according to the received different ambient temperature, control a drive current of a VCSEL light source chip, so that a wavelength of a laser beam emitted by the VCSEL light source chip is scanned at different absorption peak wavelengths, to carry out a measurement to methane concentration,
perform an inversion calculation based on a calibration value to obtain a value of the methane concentration, and
judge whether the obtained value of the methane concentration is within a preset safety range, which comprises:
if yes, to switch a state of the corresponding pulse-driven VCSEL-based methane detection module to a “sleep” mode until the end of a preset sleep period, wake up the corresponding pulse-driven VCSEL-based methane detection module once again to perform the detection of the methane concentration; and, if the obtained value of the methane concentration through the detection is always within the preset safety range, to control the pulse-driven VCSEL-based methane detection module to continuously operate alternately and repeatedly in a “wake-up” mode and the “sleep” mode;
if not, which indicates that the obtained value of the methane concentration exceeds the preset safety range, to send out an alarm prompt information.

2. The laser-based wireless methane monitoring system based on the pulse-driven VCSEL according to claim 1, wherein the pulse-driven VCSEL-based methane detection module further comprises a gas absorption cell optical path, wherein the gas absorption cell optical path comprises a chip-on-board (COB) circuit board, a collimating concave mirror, a focusing concave mirror and an elliptic concave mirror; wherein,
the VCSEL light source chip and a photodetector chip are respectively fixed at preset positions on the COB circuit board, a light outlet of the VCSEL light source chip is positioned at a focus of the collimating concave mirror, a photosensitive surface of the photodetector chip is positioned at a focus of the focusing concave mirror, and the VCSEL light source chip and the photodetector chip are further respectively connected to the MCU control module; and
a light beam emitted from the light outlet of the VCSEL light source chip is reflected by the collimating concave mirror to form a collimated light beam parallel to a plane the COB circuit board, the collimated light beam is reflected by the elliptic mirror and then is incident on the focusing concave mirror, and the incident light beam is reflected by the focusing concave mirror to focus on the photosensitive surface of the photodetector chip, and the photodetector chip converts an optical signal into an electrical signal and then transmits the electrical signal to the MCU control module.

3. The laser-based wireless methane monitoring system based on the pulse-driven VCSEL according to claim 2, wherein the pulse-driven VCSEL-based methane detection module further comprises a transistor outline-can (TO-can) device as a substitute for the VCSEL light source chip and the photodetector chip.

4. The laser-based wireless methane monitoring system based on the pulse-driven VCSEL according to claim 2, wherein the collimating concave mirror and the focusing concave mirror may be made of rotating parabolic mirror surfaces.

5. The laser-based wireless methane monitoring system based on the pulse-driven VCSEL according to claim 1, wherein the laser-based wireless methane monitoring system further comprises a monitoring terminal, wherein the monitoring terminal is connected with the laser-based methane node detection devices through a wireless gateway; and
the monitoring terminal is configured to:
receive a current ambient temperature value, the value of the methane concentration, an alarm state and an alarm node information sent from the each of the laser-based methane node detection devices at each preset time interval, and summarize, store and display the information.

6. The laser-based wireless methane monitoring system based on the pulse-driven VCSEL according to claim 1, wherein the each of the laser-based methane node detection devices further comprises a wireless data communication module and a battery driving module, wherein:
the wireless data communication module and the battery driving module are electrically connected with the MCU control module, the wireless data communication module is configured to upload data of the methane concentration to the monitoring terminal, and the battery driving module is configured to supply power to the laser-based methane node detection device.

7. The laser-based wireless methane monitoring system based on the pulse-driven VCSEL according to claim 1, wherein the each of the laser-based methane node detection devices further comprises an acousto-optic alarm module, wherein the acousto-optic alarm module is connected to the pulse-driven VCSEL-based methane detection module, and if the obtained value of the methane concentration exceeds the preset safety range, the acousto-optic alarm module makes an alarm prompt.

8. The laser-based wireless methane monitoring system based on the pulse-driven VCSEL according to claim 6, wherein the battery driving module may be a D-type battery with a model number of CR34615, and the capacity thereof is 19 Ah.

9. The laser-based wireless methane monitoring system based on the pulse-driven VCSEL according to claim 1, wherein the pulse-driven VCSEL-based methane detection module further comprises an embedded firmware, wherein the embedded firmware is written into a nonvolatile memory of the MCU control module, wherein:
after the drive current of the VCSEL light source chip is controlled, so that the wavelength of the laser beam emitted by the VCSEL light source chip is scanned at different absorption peak wavelengths, the embedded firmware may automatically search absorption peaks and perform the inversion calculation to output the value of the methane concentration.

10. A methane monitoring method by using the laser-based wireless methane monitoring system based on the pulse-driven VCSEL according to claim 1, comprising the following steps:
the MCU control module wakes up the pulse-driven VCSEL-based methane detection module corresponding thereto, to measure the ambient temperature at the current moment;
the MCU control module receives the measured ambient temperature at the current moment, then for different measured ambient temperature values, controls the drive current of the VCSEL light source chip, so that the wavelength of the laser beam emitted by the VCSEL light source chip is scanned at different absorption peak wavelengths, to carry out the measurement to the methane concentration, then performs the inversion calculation based on the calibration value to obtain the value of the methane concentration; and
the MCU control module executes a judgment that whether the obtained value of the methane concentration is in the preset safety range or not, wherein:
if yes, switches the state of the corresponding pulse-driven VCSEL-based methane detection module to the “sleep” mode until the end of a preset sleep period, wakes up the corresponding pulse-driven VCSEL-based methane detection module once again to perform the detection of the methane concentration; and, if the obtained value of the methane concentration is always within the preset safety range, controls the pulse-driven VCSEL-based methane detection module to continuously operate alternately and repeatedly in a “wake-up” mode and the “sleep” mode;
if not, which indicates that the obtained value of the methane concentration exceeds the preset safety range, controls a corresponding laser-based methane node detection device to make an alarm prompt.
